(19) United States
(12) Patent Application Publication
(10) Pub. No.: US 2008/0299227 A1
(43) Pub. Date: Dec. 4, 2008

(54) Method for removal of HIV from the gastrointestinal tract and bile of HIV-infected subjects and for the prevention of drug failure and prevention of development of AIDS in HIV-infected subjects

(75) Inventor: George M. Santamarina, Miami, FL (US)

Correspondence Address: GEORGE M SANTAMARINA, SUITE 203, 13831 SW 59 STREET, MIAMI, FL, 33183, US

(73) Assignee: SunHealth Therapies, Inc., Miami, FL (US)

(21) Appl. No.: 11/893,199

(22) Filed: Aug. 15, 2007

Related U.S. Application Data
(60) Provisional application No. 60/932,341, filed on May 30, 2007.

(51) Int. Cl. A61K 33/08; A61P 1/00; A61P 31/18

(52) U.S. Cl. 424/692

(57) ABSTRACT

The present invention discloses a novel use of magnesium oxide and magnesium hydroxide formulation in adequate proportions and sufficient amounts to induce bowel movement to induce the excretion of HIV, along with HIV-infected pathogens present in the intestinal tract of HIV-infected persons, and to induce the excretion of HIV-loaded bile from said persons. The present invention may prevent the development of drug resistance in HIV patients undergoing drug therapy, may accelerate the recovery of HIV-infected patients, may permit full recovery of these individuals, may prevent viral mutation of patients undergoing HIV drug therapy, and may prevent the onset of AIDS in HIV-infected patients. As an additional benefit, the present invention will contribute to the prevention of adipose tissue disorders, frequently seen in persons undergoing drug therapy. The present invention may be used by itself or together with HAART or other drug therapies.

### BACKGROUND OF THE INVENTION

1. Field of the Invention

The present invention is directed toward methods and formulations for treating HIV infections by inducing the exertion of HIV and HIV-infected pathogenic organisms from the intestinal tract of mammals, as well as inducing the excretion of HIV-laded bile from the subjects. The present invention is directed to prevent gastrointestinal complications in HIV-infected patients, it is further directed to prevent viral mutatior in HIV patients under HAART or other drug treatment, to prevent the development of viral resistance in these patients, and to prevent the development of AIDS in HIV-infected persons. One additional benefit of the present invention is the prevention, or at least reduction, of adipose tissue disorders.

The Human Immunodeficiency Virus (HIV) has become a major worldwide health concern which threatens with extinction a sizable portion of humanity. Despite efforts to control HIV, to this date no effective remedy has been found. Current treatments for HIV attempt to retard the progress of the infection or relieve some of its symptoms, but no cure has been found to HIV infection.

HIV infection normally leads to a progressive reduction in the immune system's capabilities, which in turn leads to numerous secondary infections and complications. It has been theorized, although not necessarily proven, that HIV is directly responsible for the development of the health complications characteristic of AIDS as a result of its attack on immune system cells.

It has been determined that upon infection by HIV, the intestinal tract becomes fertile ground for HIV replication. The intestinal mucosa harbors the majority of the body's lymphocytes, compared to the peripheral blood, which contains only 2-5% of all lymphocytes (1). The process of HIV replication in the intestinal tract is directly responsible for the reduction of the immune system cells that reside in the tract, and for the gradual reduction of these cells seen in HIV-infected subjects, even in those subjects undergoing drug therapy.

One of the parameters of greatest importance for the determination of HIV/AIDS in an HIV-infected person is the decrease in number of CD4+ cells in the HIV-infected person. This decrease is the direct result of HIV replication in the intestinal tract.

It is commonly seen in HIV-infected subjects undergoing drug therapy that even in those cases where blood tests reveal that the HIV-infected subject has undetectable levels of HIV in the blood stream. The subject continues to have very low CD4+ or other lymphocyte counts, or suffers continually decreasing counts of these culls. The reason for this discrepancy is due to the fact that present drug therapies do not restrain HIV replication in the intestinal tract, the place which in fact serves as replication platform for most of the HIV present in the HIV-infected person.

The gastrointestinal tract is a most important route of HIV entry and by far the dominant site for the HIV replication. HIV seems to multiply at a much faster rate in the intestinal tract than anywhere else in the body, and HIV seems to be present in much larger amounts in the intestinal tract than in other parts of the body. The liver collects HIV from the blood and transfers it to the bile, which subsequently is secreted into the small intestine as part of the digestive process, increasing the amount of HIV in the intestines.

Common gastrointestinal complications in HIV-infected persons are malnutrition, nutrient malabsorption, diarrhea end weight loss. (Ehrenpresis et al, Acquir Immune Defic. Syndr 5(10): 1047 1050 (1992); (Ehrenpresis et al, Am J Clin Pathol 97(1):21 28 (1992). Many persons continue to experience these complications, even years after starting treatment of HIV with current drugs.

While current HIV therapies can control HIV replication in the blood, they have little or no effect on the HIV levels in the intestinal tract, which is where most HIV replication, appears to take places, and where most of HIV probably resides. HIV appears to hide from the HIV drugs in the intestinal tract. Dandekar et al, 2006. Restoring Normal levels of CD4 T cells in the gut is not possible with present drugs.

There appear to be a number of clinical abnormalities in the gastrointestinal tract associated with HIV infection. Among these are immune suppression and pathological changes in intestinal tissues, such as inflammation, villus atrophy as well us physiological changes, such as diminished digestive enzyme activities (Heise et al, Gastroenterology 100(6):1521 1527 (1991); Ullrich et al, Ann Intern Med 111 (1):1521 (1939).

Furthermore, HIV infection does not follow the infection pattern of similar viruses. To this day, no one has been able to explain why HIV behaves in a fashion totally different to similar viruses. For example, some individuals become infected but never suffer from AIDS. Other individuals get infected and develop AIDS symptoms in a relatively short span of time, while it may take years for others to develop AIDS symptoms. This erratic behavior has led some researchers to believe that HIV is not the cause of AIDS. Indeed, this unpredictable pattern contradicts the theory that AIDS is caused solely by HIV, and favors the theory that there must be other factor or factors that influence the development of AIDS in HIV-infected persons.

It could be speculated that the peculiar behavior of HIV could somehow be related to the small size of the virus, which allows it to actually hide in other organisms that are resistant to the immune system. There are a number of organisms that: are able to exist in our body without being destroyed by the immune system. If HIV could hide in those organisms, then it could be safely assumed that those organisms could in fact be sanctuaries for HIV. This would, in turn, allow the virus to increase dramatically in number without being challenged by the immune system, as we know is the case with HIV.

Candida and parasites are two types of organisms that commonly exist in the intestinal tract. Both can remain in the intestinal tract without major interference from the immune system. HIV can easily hide in candida. Candida is a yeast that transforms itself into a pathological fungus whenever it is allowed to grow in numbers. The immune system is one of the factors that prevents candida from growing in numbers. If the immune system cells get depleted then candida can grow in number Large growths of fungal candida are common in HIV-infected persons, and specially in persons that exhibit AIDS symptoms. As the candida population increases, it covers the walls of the intestinal tract, and this body's ability to assimilate nutrients decreases accordingly, a condition typical of later stages of HIV infection and AIDS.

The present invention, by removing candida, parasites and HIV copies from the intestinal tract, may be able to postpone indefinitely the onset of AIDS in HIV-infected persons. This route holds better possibilities than present drugs, as it concentrates its efforts in destroying the bulk of the infection, present in the intestinal tract.

In order to eliminate candida and HIV successfully, any parasites present in the intestinal tract or the liver must be eliminated, because parasites can harbor both candida and HIV. Larger intestinal parasites can harbor very large amounts of candida and HIV. Smaller parasites that exist in large numbers can also harbor large amounts of candida and HIV. Parasites and pathological candida must be eliminated from the intestinal tract and the liver if HIV is to be eliminated from the intestinal tract.

If candida can harbor HIV, and parasites can harbor large amounts of candida and HIV, then if we have a method to flush out candida, parasites and HIV from the intestinal tract, we will be able to flush out HIV in large amounts from the intestinal tract and the body. This in turn, may provide a simple way to prevent the development of AIDS in HIV-infected persons.

Even if candida and parasites were not harbors for HIV, if we are able to flush out the HIV that resides in the intestinal tract, we would be able to easily accomplish a task that up to now could not be accomplished, no matter how hard it has been tried.

It is now generally accepted that the gastrointestinal tract is major route of infection of HIV. We can theorize an HIV cycle in the body as follows: Assume a healthy person comes in contact with HIV. The body will immediately put up a fight to destroy the foreign organism, which at some point becomes invisible to the body's defenses. Why can't the body's defenses destroy the virus when it first invades the body? Perhaps it can in some instances, and the person does not become infected. However, in those cases when the person does, become infected, it may be that the virus hides where it cannot be detected. One place could be inside candida.

We know that HIV has a predilection for mucous tissues. So does candida. Mucous tissues are found in the gastrointestinal tract, the sexual organs, and the upper respiratory tract. Mucous tissues are humid arid provide a nice environment for fungus to grow. The intestinal tract usually has a very large number of immune system cells, which the virus will use for its fast replication. As the number of immune cells diminishes in the intestinal tract, the amount of fungal candida will increase, and the HIV presence in the intestinal tract will continue to increase as it hides in the candida and any parasites present. After every meal, the virus is absorbed with nutrients and water into the blood and the lymph. These will distribute the virus throughout the body. The virus appears to have a larger presence in mucosal tissue, rich in blood supply and immune cells, but is present in the whole body. The liver will pick up the virus from the blood and will transfer it to the bile. The bile with virus will be secreted into the small intestine as part of the digestive process, and the cycle repeats itself. As the levels of candida increase in the intestinal tract, its roots provide larger and faster routes of entry of the virus into the body, and the cycle will increase its volume of access into the same.

Present drugs control the replication of HIV in the blood, but not in the intestinal tract. HIV drugs cannot break the HIV cycle because they don't address the bulk of the infection, which occurs in the intestinal tract. If on the other hand, we can break the HIV cycle in the intestinal tract, where most of HIV replication takes place and where most of HIV resides, by flushing out HIV, candida and parasites replete with HIV, we will have a far greater opportunity of controlling HIV than that offered by present drugs.

The HIV cycle outlined above helps to explain why drugs tend to fail in the HIV treatment and why when drug treatment is discontinued HIV may attack with greater strength than before treatment.

Suppose an HIV-infected person begins a drug treatment. The treatment is continued for some time. Natural selection tells us that the first viral copies to be eliminated or restrained would probably be the weaker ones. The stronger copies in the blood would probably make it back to the intestinal tract where they would replicate and would be reabsorbed into the blood at some point. Stronger and stronger strains of the virus will continue to develop in the intestinal tract, and the blood. At some point, the strains may overwhelm the protection of the drugs and drug failure may occur. Even if no drug failure occurs, drug therapy has the inherent characteristic of developing stronger strains. If the therapy is suspended, a stronger HIV will emerge.

One advantage of the present invention is that it may contribute to the elimination, or at least the reduction, of adipose tissue disorders, which are frequently seen in HIV-positive persons undergoing medication. This disorder id due to the destruction of HIV, causing the lipid portion of the destroyed virus to accumulate in the body. As we eliminate the volume of HIV present in the intestinal tract, there will be less copies of HIV in the blood, and less adipose tissue to accumulate in the body. As the body normalizes itself, the adipose accumulations will tend to disappear spontaneously.

The present invention may be used by itself, or in conjunction with HAART or other drug therapy to prevent drug failure, or to accelerate the progress of HIV-infected persons. It may also be used alternating with any drug therapy.

2. Description of the Related Art

The following studies point out the drastic growth of HIV in the intestinal tract, and the uncontrolled attack of the infection on the immune system in the intestines, from where the virus infiltrates the body. None of the studies provide any solution, or any guide, that may provide a solution to the uncontrolled growth of HIV in the intestinal tract. Mehandru et al, “Primary HIV-1 Infection is Associated with Preferential Depletion of DE4T Limphocytes from Effector Sites in the Gastrointestinal Tract”, The Journal of Experimental Medicine, Volume 200, Number 6, Sep. 20, 2004, 761-770. http://www.jem.org/cgi/doi/10.1084/jem.20041196. This study shows that HIV destroys many more immune system, cells in the intestines than in the rest of the body.

Also, another publication by the previous publication's author discusses HIV infection. Mehandru, et al (2007). Mechanisms of Gastrointestinal CD$+T-Cell Depletion during Acute and Early Human Immunodeficiency Virus Type 1 Infection. J. Virol. 81: 599-612. This study points out the large destruction of immune system cells in the intestines during the very early stages of HIV infection.

Another related reference corresponds to Critchfield et al (2007). Multifunctional Human Immunodefidency Virus (HIV) Gag-Specific CD8+-T-Cell Responses in Rectal Mucosa and Peripheral Blood Mononuclear Cells during Chronic HIV Type 1 Infection. J. Virol 81 5460-5471. This study shows that the immune system in rectal mucosa may be able to spontaneously destroy some infected cells.

Miyake et al (2006). Rapid dissemination of a pathogenic simian/human immunodeficiency virus to systemic organs and active replication in lymphoid tissues following intrarectal infection. J. Gen. Virol. 87: 1311-1320. This study discusses the proliferation of HIV to the body of a macaque after intrarectal inoculation of simiar HIV.

Herzberg et al (2006). (C3) The Oral Epithelial Cell and First Encounters with HIV-1. Adv. Dent. Res. 19: 158-16.

Dandekar et al (2006) Viral Suppression and Immune Restoration in the Gastrointestinal Mucosa of Human Immunodeficiency Virus Type 1-Infected Patients Initiating Therapy during Primary or Chronic Infection, Journal of Virology August 2006, p 8236-8247 Vol 80, No. 16. The study suggests possible consequences of the lack of viral suppression in the gastrointestinal during HAART therapy, such as increased immune activation and inflammation.

Kotler, Donald P (2005) HIV Infection and the Gastrointestinal Tract, Editorial Review, AIDS: Volume 19(2) 28 Jan. 2005 p 107-117.

None of the foregoing references disclose a treatment involving the excretion of HIV from the gastrointestinal tract.

### SUMMARY OF THE INVENTION

The main object of the invention relates to a method for the excretion of HIV from the gastrointestinal tract.

Another object of the invention relates to a method for achieving or promoting the excretion of entities or organisms that may harbor HIV in the gastrointestinal tract. Among these entities are different types of candida, intestinal parasites such as worms, protozoa and others, as well as fungi, viruses, and pathological bacteria.

Another object of the invention is to achieve or promote the excretion of fungi, bacteria, viruses and other entities that may cause or held to cause inflammation or other pathology of the gastrointestinal tract.

Another object of the invention is the excretion of entities or organisms that may cause or held to cause gastrointestinal complications such as diarrhea, weight loss, nutrient malabsorption and others.

Another object of the invention is the excretion of HIV-loaded bile.

Another object of the invention is the prevention of viral mutation in patients undergoing HIV drug therapy.

Another object of the invention is the prevention of the development of AIDS in HIV-infected individuals.

Another object of the invention is the reduction of adipose tissue disorders in HIV-positive persons undergoing drug therapy.

### DETAILED DESCRIPTION OF THE PREFERRED EMBODIMENT

The following is given to further illustrate the present invention. The scope of the invention is not meant to be limited to specific details hereof. In one embodiment, two teaspoons (10 g) of magnesium oxide and magnesium hydroxide powder are diluted in eight ounces of water in which two ounces of lemon or lime juice are added, and ingested in an empty stomach. As an alternative, eight capsules of the magnesium compound may be used in lieu of the powder. This embodiment may be repeated after twelve hours.

The above embodiments may be altered by reducing the dosages in half, or increasing them by one-third, depending on the specific circumstances of the HIV-infected patient, to undertake a more or less gradual cleanup of the gastrointestinal tract. An increase in the dosage will result in greater excretion of HIV-loaded bile.

The ingestion of fats after the first bowel movement will also result in greater excretion of HIV-loaded bile.

In another embodiment, once an HIV-infected person has gone through the basic embodiments described above for some time, the patient may be subjected to a supervised fast of a least 72 hours during which the patient receives any of the above embodiments at least once daily.

The above embodiments may be accompanied by special dietary regimes to exclude sugar intake and refined carbohydrates, or other dietary considerations.

All of the embodiments can be used as a self-sufficient program, or can be included as an adjuvant of a HAART or other drug regime for greater benefit, or use it alternating with any drug therapies.

While the invention has been illustrated with respect to specific dosages, it is apparent that variations and modifications can be made without departing from the spirit or scope of the invention.

What is claimed is:

1. (canceled)

2. The method according to claim 42 wherein said administration is orally.

3. The method according to claim 42 wherein a magnesium oxide and magnesium hydroxide formulation is administered in powder form dissolved in water or other liquid.

4. The method according to claim 42 wherein a magnesium oxide and magnesium, hydroxide formulation is administered in capsules.

5. The method of claim 42, wherein the formulation is administered once daily.

6. The method of claim 42, wherein the formulation is administered twice daily.

7. The method of claim 42, wherein the formulation is administered three times daily.

8. The method of claim 4, wherein said formulation is administered once daily.

9. The method of claim 4, wherein said formulation is administered twice daily.

10. The method of claim 4, wherein said formulation is administered three times daily.

11. A method of treating gastrointestinal complications, including but not limited to loss of appetite, malnutrition, nutrient malabsorption, diarrhea and weight loss associated with HIV infection comprising the step of inducing the excretion of HIV and other pathogenic organisms from the gastrointestinal tract of the person.

12. The method according to claim 11 wherein said administration is orally.

13. The method according to claim 11 wherein the step of Induction of excretion includes a sufficient amount of magnesium oxide and magnesium hydroxide formulation administered in powder form dissolved in water or other liquid.

14. The method according to claim 11 wherein the step of induction of excretion includes a sufficient amount of magnesium oxide and magnesium hydroxide formulation administered in capsules.

15. The method of claim 11, wherein the formulation is administered once daily.

16. The method of claim 11, wherein the formulation is administered twice daily.

17. The method of claim 11, wherein the formulation is administered three times daily.

18. The method of claim 14, wherein said formulation is administered once daily.

19. The method of claim 14, wherein said formulation is administered twice daily.

20. The method of claim 14, wherein said formulation is administered three times daily.

21. A method for preventing the development of HIV resistance in patients undergoing HAART or any HIV drug therapy.

22. The method according to claim 21 wherein said administration is orally.

23. The method according to claim 21 wherein a magnesium oxide and magnesium hydroxide formulation in adequate proportions to induce bowel movement is administered in powder form dissolved in water or other liquid.

24. The method according to claim 21 wherein a magnesium oxide and magnesium hydroxide formulation in adequate proportions and sufficient amounts to induce bowel movement is administered in capsules.

25. The method of claim 21, wherein the formulation is administered once daily.

26. The method of claim 21, wherein the formulation is administered twice daily.

27. The method of claim 21, wherein the formulation is administered three times daily.

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. A method for treating an HIV-infected person comprising the steps of:
A) preparing a person through fasting for a predetermined amount of time;
B) administering, at least twice per week for as long as necessary, a composition that includes a sufficient amount of a magnesium oxide and magnesium hydroxide formulation diluted with a predetermined amount of water to induce the excretior of HIV and HIV-infected pathogenic organisms from said person's gastrointestinal tract and also inducing the excretion of HIV-loaded bile from said person.
